(19) United States
(12) Patent Application Publication
(10) Pub. No.: US 2010/0074894 A1
(43) Pub. Date: Mar. 25, 2010

(54) PHARMACEUTICAL COMPOSITION CONTAINING ANTIBODIES DIRECTED AGAINST THE HERV-W ENVELOPE

(75) Inventor: Herve Perron, Saint Genis Les Ollieres (FR)

Correspondence Address: OLIFF & BERRIDGE, PLC, P.O. BOX 320850, ALEXANDRIA, VA, 22320-4850, US

(73) Assignee: GENEURO SA, PLAN-LES-OUATES (CH)

(21) Appl. No.: 12/449,327

(22) PCT Filed: Feb. 11, 2008
(86) PCT No.: PCT/FR2008/000166

§ 371 Date: Oct. 26, 2009

(30) Foreign Application Priority Data
Feb. 9, 2007 (FR) 0700952

(51) Int. Cl. A61K 39/395; A61P 31/12; C07K 16/08

(52) U.S. Cl. 424/133.1; 424/159.1; 530/387.1

(57) ABSTRACT

A pharmaceutical composition that contains, as an active ingredient, at least one antibody directed against the HERV-W envelope protein, except for any antibody specifically directed against the binding site between said env protein and the hASCT1 or hASCT2 receptor.

### BACKGROUND OF THE INVENTION

Human endogenous retroviruses (HERVs) constitute 8% of the human genome and are involved both in pathologies and in nonpathological phenomena.

The human endogenous retrovirus W (HERV-W) family is derived from an infectious retroviral element integrated into the germ line, 25 to 40 million years ago. The envelope protein of HERV-W, also called syncytin, is a fusogenic glycoprotein involved in the formation of the syncytiotrophoblastic layer of the placenta. It is encoded by the Env gene of the ERVW1 proviral locus and synthesized in the form of a precursor, gPr73, which is specifically cleaved into two mature proteins, a surface subunit gp50 (SU) and a transmembrane subunit gp24 (TM).

In vitro, syncytin of the HERV-W family induces a cell-to-cell fusion that is dependent on its interaction with a receptor-transporter of amino acids of the ASCT family (h-ASCT2, hASCT1). Phylogenic studies then showed that syncytin is related to a group of retroviruses comprising in particular the cat endogenous virus RD114, the monkey endogenous virus BaEV, simian retroviruses and avian retroviruses: avian reticuloendotheliosis virus REV-A, and spleen necrosis virus SNV, all having in common the type 2 sodium-dependent neutral amino acid receptor-transporter or hASCT2 (Rasko et al, 1999, Proc. Natl. Acad. Sci. USA Vol. 96, pp. 2129-2134; Tailor et al, 1999 JOURNAL OF VIROLOGY, VOL 73(5) May 1999, p. 4470-4474). Thus, the infection of cells with viruses of this group of retroviruses results in a specific reduction in amino acid transport (Rasko et al, 1999). The infection of a cell with one of these retroviruses (or the expression of one of these envelopes in the cell) prevents, through interference (interaction) in relation to a receptor of the ASCT family, the infection of this same cell with another of these retroviruses or the fusion with another cell expressing another envelope. Through interference in relation to a receptor of the ASCT family, the infection of a cell with one of these retroviruses prevents infection with another of these retroviruses. All these retroviruses belong to the same HERV-W virus interference group.

The mechanisms of binding between the envelope and the ASCT receptor remain obscure. This theme is nevertheless essential since the inhibition of the envelope/ASCT receptor interaction would in addition make it possible to prevent the entry of a retrovirus into the cell, and therefore to block its replication cycle, to block the phenomenon of envelope/ASCT receptor interaction and/or of cell fusion which may be involved in the formation of tumors, in the proliferation of metastatic cells or in drug resistance phenomena (see, by way of illustration, the publication “Cell fusion: A hidden enemy? Cancer Cell: May 2003 Vol. 3), to block the phenomenon of envelope/ASCT receptor interaction and/or of cell fusion which may be involved in nervous system diseases, or even to inhibit the cell-cell fusion involved in trophoblastic differentiation. Furthermore, the inhibition of the envelope/hASCT receptor interaction could prevent tumor propagation by counteracting a local immunosuppression that may result from the envelope/hASCT receptor interaction. Indeed, it has been shown, on the one hand, that the infection of cells with viruses of this group of retroviruses (in particular those inducing immunodeficiencies) leads to a specific reduction in amino acid transport (Rasko et al, 1999), and on the other hand, a direct link is proposed between the impairment of the amino acid transport and immunosuppression (Espinosa et al., 2000; Rasko et al., 1999). Thus, as regards nervous system diseases, it is known that hASCT receptors are involved in the specific transport of neutral amino acids and that neuronal cells, predominantly use neuromediators of polypeptide nature to transmit information. Thus, the binding of the Env-HERV-W protein to receptors which normally have to transport the amino acids required for neuromediator synthesis can affect the ability of neurons to synthesize neuromediators by reducing the entry of the physiological agonists, namely the amino acids, via the ASCT receptors. Moreover, if neurons whose intercellular networks form connections which are essential for the transmission of information circulating in the brain and the spinal cord form syncytia following fusion of several neurons, induced by the Env-HERV-W protein, all the networks for transmission of information become disrupted and connected to the same fused “cellular package” and, furthermore, the neuromediator production activity of each cell is no longer individualized or connected to the upstream or downstream conduction pathways (dendrites and axons) which are specific thereto.

It has now been shown, surprisingly, that antibodies which are not specifically directed against the site for binding between the HERV-W envelope protein and an hASCT-type receptor, in particular the hASCT1 or hASCT2 receptor, are capable of blocking the interaction of the HERV-W envelope protein and an hASCT-type receptor, in particular the hASCT1 or hASCT2 receptor, and/or of inhibiting the cell fusion induced by said protein.

### SUMMARY OF THE INVENTION

Thus, a subject of the present invention is a therapeutic composition comprising, as an active substance or an active ingredient, at least one antibody directed against the HERV-W envelope protein, except for any antibody specifically directed against the site for binding between said Env protein and the hASCT1 or hASCT2 protein, and a pharmaceutically acceptable carrier.

The term “pharmaceutical composition” is intended to mean a diagnostic, prophylactic or therapeutic composition.

Said composition or said antibody at least is capable of inhibiting the interaction between the envelope protein and an hASCT-type receptor and/or the fusogenic properties of said protein.

According to one variant of the invention, said antibody is chosen from the antibodies directed against the glycosylated or nonglycosylated C-terminal end of the SU region of said envelope protein and the antibodies directed against the transmembrane (TM) region of said envelope protein. One of the suitable antibodies which are directed against the glycosylated or nonglycosylated C-terminal end of the SU region of the HERV-W envelope protein is the monoclonal antibody MoAb2 (also known as 1F11B10). In combination or as a variant, one of the suitable antibodies which are directed against the transmembrane region of the HERV-W envelope protein is the polyclonal antibody PolAb2 (also referenced 71).

Given their properties, the above antibodies find, according to the invention, the following applications.

Thus, the following uses are part of the invention:

use of at least one of said antibodies for preparing a pharmaceutical composition for the treatment of pathologies associated with an interaction between the HERV-W envelope and an hASCT receptor;
use of at least one of said antibodies for preparing a pharmaceutical composition for treating pathologies associated with the pro-inflammatory cascade induced by the expression of MSRV/HERV-W;
use of at least one of said antibodies for preparing a pharmaceutical composition for inhibiting the cell fusion induced by the HERV-W Env protein;
use of at least one of said antibodies for preparing a pharmaceutical composition for inhibiting the binding between said Env protein and an hASCT-type receptor, such as the hASCT1 or hASCT2 receptor, at the surface of the cells which express said receptor.

The abovementioned pathologies are nervous system pathologies or neuropsychiatric pathologies. By way of illustration, mention may be made of the article by S. Weis et al., J Neural Transm. 2007 February; 114(2):261-71, which associates an impairment of the expression of the abovementioned receptors in schizophrenia, manic depressive psychosis and major depressions. Thus, the invention relates to the uses of at least one antibody in the treatment of said pathologies.

According to variants of use according to the invention, when the antibody is directed against the C-terminal end of the SU region of the HERV-W envelope protein, it is preferably the monoclonal antibody MoAb2; when the antibody is directed against the transmembrane region of the HERV-W envelope protein, it is preferably the polyclonal antibody PolAb2.

Another subject of the invention is an antibody directed against the HERV-W envelope protein, except for any antibody specifically directed against the site for binding between said Env protein and an hASCT-type receptor, such as the hASCT1 or hASCT2 receptor, capable of inhibiting the cell fusion induced by the HERV-W Env protein and/or of inhibiting the binding between the HERV-W envelope protein and an hASCT-type receptor, such as the hASCT1 or hASCT2 receptor.

A preferred antibody is directed against the glycosylated or nonglycosylated C-terminal end of the SU region of the HERV-W envelope protein. It can be obtained from a hybridoma derived from splenocytes of mice immunized with the HERV-W envelope gene, according to techniques which are part of the general knowledge of those skilled in the art. Such an antibody may consist of the monoclonal antibody MoAb2. Advantageously, said antibody is humanized.

Another preferred antibody is directed against the transmembrane region of the HERV-W envelope protein. Such an antibody may consist of the polyclonal antibody PolAb2.

### EXAMPLE 1

### Molecular and Phenotypic Characterization of Recombinant Envelopes

### Construction and Production of the HERV-W Envelope Subunit SU

On the basis of the expression vector phCMV-Env-W (Blond J Virol, Vol 74(7):3321-3329, 2000) containing the gene of the HERV-W envelope (538 amino acids) (clone PH74, Blond et al J Virol Vol 73(2):1175-1185, 1999); a vector, phCMVEnv-Gp60, which allows the expression of a soluble recombinant envelope protein, was designed.

The soluble envelope (Gp60, 1-435) was constructed as described below:

(1) the native cleavage site RNKR (AA 314 to 317) between the SU and TM subunits was mutated to AAAR, in order to allow the production of a stable fusion protein, and not of two SU-TM subunits cleaved and then re-associated via a disulfide bridge.
(2) The transmembrane (TM) and intracytoplasmic (CYT) regions corresponding to amino acids 436 to 538 were deleted in order to obtain a soluble protein.
(3) A spacer arm having the composition (GGGS)3 followed by a polyhistidine tail (RGS-HHHHHH) were added at the C-terminal position, so as to enable purification of this protein by IMAC and detection with an anti-histidine monoclonal antibody (Qiagen, RGS H6).

The vector phCMV-EnvSU was constructed from the vector phCMVEnv-Gp60 expressing the soluble envelope, thereby enabling the production of an SU protein. The soluble SU is a fusion protein containing a C-terminal polyhistidine tail having the sequence RGS-HHHHHH immediately downstream of the AAAR sequence so as to enable purification of this protein by IMAC and detection with an antihistidine monoclonal antibody (Qiagen, RGS H6).

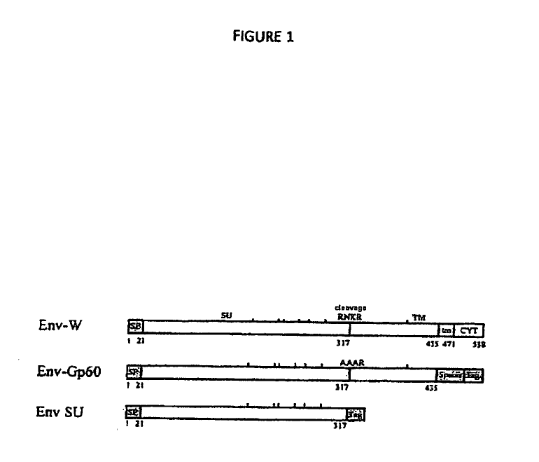

The schematic structure of the different proteins produced from the phCMV-Env-W, phCMV-EnvGp60 and phCMV-EnvSU vectors is illustrated in FIG. 1.

### EXAMPLE 2

### Obtaining a Monoclonal Antibody Directed Against the HERV-W Envelope Protein

Immunization of Mice with DNA

Three six-week-old female BALB/c mice (IFFA-Credo) were immunized by direct injection of naked plasmid DNA (phCMV-Env-W) containing the HERV-W envelope gene. The injections were carried out intradermally using a gene gun. Five injections of 2 μg of DNA were first given for each mouse, followed by a booster with two injections of 4 μg of DNA. The sera were sampled and the antibody titer for each serum was determined. Since the antibody titer was too low, a cell lysate was prepared.

### Preparation of the Cell Lysate

TelCeB6 rhabdomyosarcoma cells (ATCC CRL8805) were transfected with the phCMV-Env-W plasmid. After about 20 hours in the presence of syncytia, a cell extract was prepared in PBS buffer containing 0.5% triton. The protein extracts were assayed by the Bradford method. The concentration of Env-W antigen corresponded to 9.5 μg/μl of total proteins.

Immunization of Mice with Cell Lysate Extract

The same mice were first of all given an injection of 10 μg of cell lysate intraperitoneally, followed by a booster injection of 2×100 μg of cell lysate intraperitoneally. Three days before the fusion with the myeloma cells, a further injection was carried out, intravenously, this injection being 22 μg of the soluble envelope protein Gp60 obtained from the phCMV-Env-Gp60 plasmid as described in example 1, purified beforehand, before injection, on an Ni-NTA resin (Qiagen) according to the following conditions: binding in phosphate buffer, pH 8, washing in phosphate buffer, pH 8 and in ammonia acetate, pH 6, elution in ammonium acetate buffer, pH 3.5, and concentration on a speed vac. 47 μg of the eukaryotic Gp60 protein thus obtained were reserved for the intravenous injection described above. After fusion, the hybridoma supernatants were tested by immunofluorescence on the transfected and fixed cells (TelCeb6), the antibodies were screened by means of a functional ELISA assay using the Env-W protein at a concentration of 9.2 μg/μl of total proteins and an Env AS protein as negative control at a concentration of 13.3 μg/μl of total proteins, and the most effective antibodies were selected.

The monoclonal antibody MoAb2 was thus obtained. It is a monoclonal antibody directed against the C-terminal part of the SU region of the Env-HERV-W protein.

### EXAMPLE 3

### Study, in an Animal Model A1, of the In Vivo Formation of Syncytia when Susceptible Cells are Transfected with Plasmids Encoding Various Env Proteins of the MSRV/HERV-W Family and of the Inhibition of the Formation of such Syncytia when Antibodies Directed Against These MSRV/HERV-W Env Proteins are Injected

### 3.1 Materials:

Cells in a 100 mm-diameter dish at confluence
LipofectAMINE PLUS™ kit (Gibco Invitrogen)
DMEM medium (Gibco Invitrogen 41966-029) with South America serum
TelCeB6 cells (ATCC CRL8805-rhabdomyosarcoma cells)
DNAs: 409 (W envelope cloned in the sense direction) 2 μg/μl
410 (W envelope cloned in the antisense direction) 1.5 μg/μl
LQMV (nonfusogenic mutated envelope) 1.3 μg/μl.

### 3.2 Protocol:

### Day 1: Cells Placed in Culture

Inoculation of 100 mm-diameter dishes
50-70% confluence for the TelCeB6
Incubation in supplemented medium (6 ml per dish) for 24 h at 37° C. under 5% CO₂.

### Day 2: Transfection—LipofectAMINE PLUS™ kit

### a) Precomplexation of the DNA

mixing of 750 μl of nonsupplemented medium with the DNAs in a 15 ml falcon tube (reference 2096)
either 2 μl of 409 or 3 μl of 410 or 3 μl of LQMV
vortexing of the PLUS Reagent and addition of 20 μl thereof to the DNA solution
immediate vortexing for 10 sec at 1400 rpm
incubation for 15 min at ambient temperature.
   b) Preparation of the cells
replacement with 5 ml of nonsupplemented medium.
   c) Dilution of the lipofectamine
In a tube and for one dish, mixing of 30 μl of LipofectAMINE Reagent with 750 μl of nonsupplemented medium.

### d) Complexation of the DNA

Mixing of the 780 μl of diluted lipofectamine and of the 772 μl of the pre-complexed DNA solution
immediate vortexing for 10 sec at 1400 rpm
incubation for 15 min at ambient temperature.
   e) Transfection and obtaining of recipient animals grafted with the target cells, treated or not treated by injection of anti-Env antibodies
Depositing of the 1552 μl in a dish
incubation for 2-3 hours at 37° C. under 5% CO₂
replacement of the transfection medium with 6 ml of supplemented medium
incubation for 1 h at 37° C. under 5% CO₂
intraperitoneal (IP) injection in SCID mice (in a volume of 1 ml), of ⅕^{th} of each dish at 70% confluence, with or without additional injection of antibodies against MSRV/HERV-W Env proteins (antibodies: MoAb2, PolAb2, 69 and 71 at 1/100).
Obtaining of animals that tolerate the graft and allow dissemination of the grafted cells in the organism, parallel to the establishment of a pseudoascites in the peritoneal cavity.

### Day 3:

Sampling of the cells of each animal by peritoneal lavage: injection first of 2 ml of air followed by 2 ml of physiological saline, then massaging and recovery of 2 ml of peritoneal fluid (original protocol developed for recovery in animals grafted with cells implanted in the peritoneal cavity).
Observation under an “inverted phase” microscope with counting of syncytia and/or after staining on a slide.
Immediate reading. The reading is carried out after plating out on squared-chamber slides in the presence of Trypan blue (exclusion of dead cells). The number of cells having fused with one another per squared field is counted with a “large field” objective (40) making it possible to establish the count over more than about 100 cells so as to have statistically representative counting series.

A cell aliquot of each sample is fixed in the presence of methanol/acetone (v/v) and then stored at −20° C. until staining with crystal violet (1%). Photographs of these stained slides were taken.

3.3 Number of mice:
Batches of two mice will be inoculated with:
The cells transfected with the three types of plasmid (2 coding and one antisense as a control) without antibodies (3×2=6 mice).
The three types of transfected cells and the monoclonal antibody MoAb2 (3×2=6 mice).
The three types of transfected cells and an anti-TM polyclonal antibody, PolAb2 (3×2=6 mice).

### 3.4 Results:

The results obtained are reproduced in table 1 below:

Table 1 of results on the animal model A1
ECP READING (Trypan Blue*): Direct reading (syncytia)
(number of fused cells, reading on a squared counting
chamber per 100 cells)

| | Count | Count | |
|---|---|---|---|
| Lines | M1* | M2* | Average |
| 409cont. | 19 | 22 | 22 |
| 409MoAb2 | 1 | 3 | 2 |
| 409PolAb2 | 9 | 9 | 9 |
| 410cont. | 8 | 11 | 9.5 |
| 410MoAb2 | 3 | 5 | 4 |
| 410polAb2 | 9 | 14 | 11.5 |
| LQMVcont. | 8 | 5 | 6.5 |
| LQMVMoAb2 | 4 | 2 | 3 |
| LQMVPolAb2 | 8 | 4 | 6 |

*M1, M2 = mouse 1, mouse 2
Trypan Blue: Exclusion of dead cells

Each number represents the number of cells visualized as fused per field studied. Since some cells may be superimposed in the optical path, the count for the cells appearing to be fused under the control conditions is therefore greater than zero. Subsequent staining of the cells on slides, with visualization of multiple cell nuclei included in a space delimited by the continuity of one and the same cell membrane made it possible to verify that the syncytia were real and could be distinguished from stacks of cells. Moreover, upon analysis by phase contrast microscopy, photographs showing cells undergoing fusion, and the complete absence of an equivalent phenomenon in the controls, make it possible to objectify the real nature of the fusion.

However, in order to statistically objectify the primary analysis represented by the figures indicated in the table of results of the animal model A1, we performed a Chi-2 test in order to compare the data recorded under the conditions where (i) the Env protein is correctly expressed (409) versus the controls where it is not expressed (410) or is mutated so as to reduce its fusogenic effect (LQMV), and (ii) where the protein is correctly expressed (409), without injection of anti-Env antibodies versus injection of various antibodies.

Thus, the results of the statistical analysis which takes into account the “background noise” of the primary reading, without secondary analysis after staining on slides or searching for typical cells undergoing fusion (which are never seen in the controls), are the following.

### Statistical Validation of the Specificity of the Pathogenic Effect In Vivo:

Env expressed (409): 22 positives counted on average on 100 cells.
Env antisense (410 not expressed): 9.5 positives counted on average on 100 cells.
Env mutated (LQMV activity suppressed): 6.5 positives counted on average on 100 cells.
Average of the controls (410 and LQMV): 9.5+6.5/2=8%
1) Env versus control 410: Chi-2=5.89 (p<0.02)
2) Env versus control LQMV: Chi-2—9.83 (p<0.002)
3) Env versus all control (410 and LQMV): Chi-2=7.69 (p<0.01)
4) Control 410 versus control LQMV: Chi-2=0.61 (difference not significant).

The controls are therefore indeed statistically equivalent and there is no “real” difference related to the type of control.

The results obtained as early as this stage of the analysis (by not excluding the background noise associated with the artefactual images and by comparing the two types of controls with one another, which prove to be equivalent) are statistically very significant (overall p<0.01). The subsequent analyses, by staining, of the specificity of the effects therefore mainly confirm the specificity of the effect obtained in vivo in the presence of the Env protein, thus validating the animal model A1 for the production and the study, in vivo, of syncytia of which the fusion has been induced by HERV-W Env.

### Statistical Validation of the Therapeutic Activity of the Antibodies Tested on the Pathogenic Effect In Vivo:

Env expressed (409)+monoclonal antibody MobAb2 (409MoAb2): 2 positives counted on average on 100 cells.
Env expressed (409)+polyclonal antibody 71 (409PolAb2): 9 positives counted on average on 100 cells.
1) Env alone versus injection antibody MoAb2: Chi-2=18.94 (p<0.001).
2) Env alone versus injection antibody PolAb2: Chi-2=6.45 (p<0.05).

The results obtained show a statistically very significant effect for the monoclonal antibody (probability of result being random (p) and overall much lower than 0.001). (Chi-2=18.94). The subsequent analyses, by staining, of the specificity of the effects confirm, here also, the specificity of the effect obtained in vivo in the presence of the Env protein and of antibodies, thus validating the therapeutic effect on the animal model A1.

### EXAMPLE 4

### In Vivo Study, in an Animal Model A2, of the Binding of the MSRV/HERV-W Env Proteins to Cells Having Type-1 or -2 ASCT Receptors or not Having them, and of the Inhibition of this Binding at the Level of the Plasma Membrane by Injection of Antibodies Directed Against the MSRV/HERV-W Env Proteins

### 4.1 Materials:

Soluble protein: supernatant filtered through 0.45 μm containing the soluble protein (293T transfected with the plasmid 460 (envelope-spacer-His6)). Expression verified by Western blotting with an anti-RGS-His antibody.
The antibodies:
   rabbit anti-SU polyclonal antibody (anti-peptide) (69)
   rabbit anti-TM polyclonal antibody (anti-peptide) (71).
The cells: XChASCT2 cellular clone XC (ATCC CCL-165, rat cells) expressing the hASCT2 receptor.
DMEM medium (Gibco Invitrogen 41966-029) with South America serum.
Preincubation, incubation, labeling in a 1.5 ml Eppendorf tube.

### 4.2 Protocol:

a) IP inoculation of XChASCT1 cells, XChASCT2 cells and XC control cells (ASCT-) in SCID mice

Injection into mice of ⅕^{th} of a flask at 70% confluence in a volume of 2 ml.

### b) Preincubation

Incubation of the soluble-protein supernatant (filtered supernatant of the 293T line) with the antibodies (2H1H8, 1F11B10, 69 and 71),
990 μl of supernatant with 10 μl of antibody (diluted to 1/100),
1 hour at 37° C. in the cell incubator with agitation from time to time (every 15 minutes).

Inoculation of the proteins alone or with antibodies, at IP, in mice grafted with the cells (1×10⁶ cells per point, i.e. ⅕ of a confluent θ 100 mm dish).

After injection of the antibodies (200 microliters) they are maintained at IP, for 6 hours, with peritoneal massage from time to time (every 30-60 minutes).

c) Recovery of the cells by peritoneal lavage of the grafted mice
Centrifugation at 3000 rpm for 5 minutes at +4° C.
Recovery of the cell pellet and dilution in the labeling media (maintaining at +4° C. until fixing).

### d) Labeling

Primary antibody:
   Pellet taken up with 100 μl of anti-RGS-His antibody ( 1/100^{th} dilution, Qiagen) in a PBA buffer (PBS with 2% fetal calf serum and 0.1% sodium azide), maintained at +4° C.
   1 hour in ice with agitation from time to time (every 15 minutes).
   Washing in PBS buffer (1 ml per tube), maintained at +4° C.
Secondary antibody:
   Centrifugation at 3000 rpm for 5 minutes at +4° C.
   Pellet taken up with 100 μl of anti-mouse-FITC antibody (dilution to 1/20-DAKO, reference: F0479) in a PBA buffer), maintained at +4° C.
   1 hour in ice with agitation from time to time (every 15 minutes).
   2 washes in PBA buffer (1 ml per tube), maintained at +4° C.
   Pellet taken up with 500 μl of PBA, maintained at +4° C., and analysis by FACS. Alternatively, analysis by IF after fixing on slides in acetone/methanol (50%/50%) at −20° C. and counterstaining with Evans blue.
      4.3 Number of mice:

Batches of 2 mice will be inoculated with:
Each type of cells (expressing the two types of ASCT1 and ASCT2 receptors and one not expressing said receptors, for a control) without antibody (3×2=6 mice).
The three types of cells with the Env protein and the monoclonal en antibody (3×2=6 mice).
The three types of cells with the Env protein and an anti-TM polyclonal antibody (3×2=6 mice).

### 4.4 Results:

The results obtained are reproduced in table 2 below:

Table 2 of the results of the animal model A2
IF READING (microscope)
(number of fluorescent cells/total in the same field,
number of cells indicated in the table)

| | No. fluorescent | |
|---|---|---|
| Line | cells/total | Means |
| Xcont. | 1/18, 0/10 = | 1/28 |
| XMoAb2 | 0/10, 0/12, 1/16 = | 1/38 |
| XPolAb2 | 3/25, 2/40 = | 5/65 |
| ASCT1cont. | 12/40, 3/12, 9/25 = | 24/77 |
| ASCT1MoAb2 | 1/50, 0/30 = | 1/80 |
| ASCT1PolAb2 | 6/37 = | 6/37 |
| ASCT2cont. | 8/22, 15/35 = | 23/57 |
| ASCT2MoAb2 | 3/28 = | 3/28 |
| ASCT2PolAb2 | 2/36 = | 2/36 |

Each number represents the number of cells visualized as fluorescent per field studied. Since some cells may have bound fluorescents nonspecifically, the count of cells appearing to be fluorescent under the control conditions is therefore greater than zero in one of the two fields counted (mean of the two fields= 1/28, i.e. 0.036%, which is entirely correct for the background noise of such a reading technique). The real nature of the cells having bound Env protein on their ASCT1 or 2 receptor was subsequently verified by cytofluorometric analysis.

In order to statistically objectify the analysis given in the results table for the animal model A2, a Chi-2 test was carried out so as to compare the data reported under the conditions where:

(i) the Env protein can bind to an ASCT1 receptor (ASCT1cont) or an ASCT2 receptor (ASCT2cont) present at the surface of the cells grafted in SCID mice versus the grafted control cells which have no receptor (Xcont) and, as a result, onto which the Env protein injected into the corresponding animals cannot bind and does not give membrane fluorescence in the presence of an anti-Env antibody;
and (ii) where the Env protein can bind to an ASCT1 receptor (ASCT1cont) or an ASCT2 receptor (ASCT2cont) present at the surface of the cells grafted in SCID mice versus injection of antibody.

Thus, the results of the statistical analysis are the following.

### Statistical Validation of the Specificity of the Pathogenic Effect In Vivo:

ASCT1cont: 24 positives counted on average on 77 cells.
ASCT2cont: 23 positives counted on average on 57 cells.
ASCT negative cells (Xcont): 1 positive counted on average on 28 cells.
   1) Env+ASCT1 grafts versus control ASCT NEGATIVE Xcont:
      Chi-2=8.62 (p<0.01).
   2) Env+ASCT2 grafts versus control ASCT NEGATIVE Xcont:
      Chi-2=12.53 (p<0.001).
   3) Env+ASCT1 grafts versus Env+ASCT2 grafts:
Chi-2=1.21 (difference not significant). The cells expressing the ASCT1 or ASCT2 receptors at their surface are therefore clearly statistically equivalent and there is no difference in binding of Env to the subtype-1- or subtype-2-related receptor, under the conditions of this animal model A2.

The results obtained with the animals grafted with the cells expressing the ASCT1 or ASCT2 membrane receptors are statistically very significant from the viewpoint of the results obtained with the control animals grafted with the cells expressing none of these receptors at their surface. However, although no statistical difference is demonstrated between the ASCT1+ and ASCT2+ models, the difference in number of positive cells between the ASCT2 model and the control is greater for ASCT2+ (p<0.001).

These results confirm the specificity of the effect obtained in vivo in the presence of the Env protein in the A2/ASCT1+ and A2/ASCT2+ models.

### Statistical Validation of the Therapeutic Effect of the Antibodies Tested on the Pathogenic Effect In Vivo:

A2/ASCT1+ model
Env+ASCT1 grafts: 24 positives counted on average on 77 cells.
Env+ASCT1 grafts+monoclonal antibody MobAb2 (MoAb2): 1 positive counted on average on 80 cells.
Env+ASCT1 grafts+polyclonal antibody 71 (PolAb2): 6 positives counted on average on 37 cells.
1) Env+ASCT1 grafts alone versus injection of antibody MoAb2: Chi-2=26.23 (p<0.001).
2) Env+ASCT1 grafts alone versus injection of antibody.
3) Env+ASCT1 grafts alone versus injection of antibody PolAb2: Chi-2=2.88 (difference not significant).

The results obtained show a statistically very significant effect for the monoclonal antibody (probability of result being random (p) and overall much lower than 0.001) (Chi-2=26.23). Those obtained with the polyclonal antibody are not significant. The analyses confirm, here also, the specificity of the effect obtained in vivo in the presence of the Env protein and of antibody, thus validating the therapeutic effect on the animal model A2/ASCT1+ with monoclonal antibodies. However, largely unlike the animal model A1, unexpectedly, only monoclonals are effective for inhibiting the binding of the HERV-W Env protein injected into the animal in extracellular protein form.

A2/ASCT2+ model
Env+ASCT2 grafts: 23 positives counted on average on 57 cells.
Env+ASCT2 grafts+monoclonal antibody (409MoAb2): 3 positives counted on average on 28 cells.
Env+ASCT2 grafts+polyclonal antibody (409PolAb2): 2 positives counted on average on 36 cells.
   1) Env+ASCT2 grafts alone versus injection of antibody MoAb2:
      Chi-2=7.77 (p<0.01).
   2) Env+ASCT2 grafts alone versus injection of antibody PolAb2:
      Chi-2=13.59 (p<0.001).

The results obtained show a statistically very significant effect for the monoclonal antibody (probability of the result being random (p) and overall below 0.01). Those obtained with the polyclonal antibody are different than the results obtained previously with the animal model grafted with the ASCT1+ cells: the polyclonal antibody results showing a clearly significant effect (p<0.001) on the binding to the ASCT2 receptor. The effect obtained in vivo in the presence of the Env protein and of antibodies thus validates the therapeutic effect on the animal model A2/ASCT2+.

Thus, in having validated the animal models A1 and A2 (A2/ASCT1+ and A2/ASCT2+) against the appropriate controls, it was found that monoclonal antibodies tested on these animal models have a potential therapeutic activity by very significantly inhibiting the pathogenic effects of the HERV-W Env protein.

1. A pharmaceutical composition containing, as an active ingredient, at least one antibody directed against the HERV-W envelope protein, except for any antibody specifically directed against the site for binding between said Env protein and the hASCT1 or hASCT2 receptor, and a pharmaceutically acceptable carrier.

2. The composition as claimed in claim 1, wherein the antibody is chosen from the antibodies directed against the glycosylated or nonglycosylated C-terminal end of the SU region of said envelope protein and the antibodies directed against the transmembrane region of said envelope protein.

3. The composition as claimed in claim 1, wherein the antibody is directed against the glycosylated or nonglycosylated C-terminal end of the SU region of the HERV-W envelope protein.

4. The composition as claimed in claim 3, wherein said antibody can be obtained from a hybridoma derived from splenocytes of mice immunized with the HERV-W envelope gene.

5. The composition as claimed in claim 1, wherein the antibody is directed against the transmembrane region of the HERV-W envelope protein.

6. The composition as claimed in claim 1, wherein said antibody is humanized.

7. A method for treating nervous system pathologies or neuropsychiatric pathologies associated with the interaction of an HERV-W envelope and of an hASCT receptor, the method comprising administering to a patient in need thereof a pharmaceutical composition comprising at least one antibody directed against the HERV-W envelope protein, except for any antibody specifically directed against the site for binding between said Env protein and the hASCT1 or hASCT2 receptor.

8. A method for treating nervous system pathologies or neuropsychiatric pathologies associated with the pro-inflammatory cascade induced by the expression of MSRV/HERV-W, the method comprising administering to a patient in need thereof a pharmaceutical composition comprising at least one antibody directed against the HERV-W envelope protein, except for any antibody specifically directed against the site for binding between said Env protein and the hASCT1 or hASCT2 receptor.

9. The method as claimed in claim 7, wherein said pharmaceutical composition is for treating a pathology chosen from schizophrenia, manic depressive psychosis and major depressions.

10. The method as claimed in claim 7, wherein the antibody is chosen from the antibodies directed against the glycosylated or nonglycosylated C-terminal end of the SU region of said envelope protein and the antibodies directed against the transmembrane region of said envelope protein.

11. The method as claimed in claim 7, wherein the antibody is humanized.

12. An antibody directed against the HERV-W envelope protein, except for any antibody specifically directed against the site for binding between said Env protein and the hASCT1 or hASCT2 receptor, said antibody being chosen from the antibodies directed against the glycosylated or nonglycosylated C-terminal end of the SU region of said envelope protein and the antibodies directed against the transmembrane region of said envelope protein.

13. The antibody as claimed in claim 12, wherein it is directed against the glycosylated or nonglycosylated C-terminal end of the SU region of the HERV-W envelope protein.

14. The antibody as claimed in claim 13, which can be obtained from a hybridoma derived from splenocytes of cells of mice immunized with the HERV-W envelope gene.

15. The antibody as claimed in claim 12, wherein it is humanized.

16. The antibody as claimed in claim 12, wherein it is directed against the transmembrane region of the HERV-W envelope protein.
